(19) United States
(12) Patent Application Publication
(10) Pub. No.: US 2009/0287290 A1
(43) Pub. Date: Nov. 19, 2009

(54) Delivery Systems and Methods of Implantation for Prosthetic Heart Valves

(75) Inventors: Patrick E. Macaulay, Windsor, CA (US); Robert J. Murray, Santa Rosa, CA (US); Trevor Greenan, Santa Rosa, CA (US); Timothy R. Ryan, Shorewood, MN (US); Charles Tabor, Shoreview, MN (US); Matthew J. Birdsall, Santa Rosa, CA (US); Gianfranco Pellegrini, Santa Rosa, CA (US)

Correspondence Address: MEDTRONIC, INC., 710 MEDTRONIC PARKWAY NE, MINNEAPOLIS, MN, 55432-9924, US

(73) Assignee: Medtronic, Inc., Minneapolis, MN (US)

(21) Appl. No.: 12/358,084

(22) Filed: Jan. 22, 2009

Related U.S. Application Data
(60) Provisional application No. 61/062,207, filed on Jan. 24, 2008.

(51) Int. Cl. A61F 2/06

(52) U.S. Cl. 623/1.11; 623/1.23

(57) ABSTRACT

A delivery system for delivering a stented prosthetic heart valve to a lumen of a patient, the delivery system including a tubular body having a proximal end, a distal end, and a base portion with a plurality of extending elements, wherein each of the extending elements is engageable with a portion of a stent of a prosthetic heart valve. The delivery system further includes a sleeve having an inner area. The sheath is longitudinally moveable relative to the base portion from a first position where the inner area of the sleeve at least partially covers the extending elements of the base portion to a second position where the extending elements are not positioned within the inner area of the sleeve.

### CROSS-REFERENCE TO RELATED APPLICATION

The present application claims priority to U.S. Provisional Application No. 61/062,207, filed Jan. 24, 2008, and titled “Delivery Systems and Methods of Implantation for Prosthetic Heart Valves”, the entire contents of which is incorporated herein by reference in its entirety.

### TECHNICAL FIELD

The present invention relates to prosthetic heart valves. More particularly, it relates to delivery systems for percutaneously implanting prosthetic heart valves comprising a stent.

### BACKGROUND

Diseased or otherwise deficient heart valves can be repaired or replaced using heart valve surgery. Typical heart valve surgeries involve an open-heart surgical procedure that is conducted under general anesthesia, during which the heart is stopped while blood flow is controlled by a heart-lung bypass machine. This type of valve surgery is highly invasive and exposes the patient to a number of potentially serious risks, such as infection, stroke, renal failure, and adverse effects associated with use of the heart-lung machine, for example.

Recently, there has been increasing interest in minimally invasive and percutaneous replacement of cardiac valves. Such surgical techniques involve making a very small opening in the skin of the patient into which a valve assembly is inserted in the body and delivered to the heart via a delivery device similar to a catheter. This technique is often preferable to more invasive forms of surgery, such as the open-heart surgical procedure described above. In the context of pulmonary valve replacement, U.S. Patent Application Publication Nos. 2003/0199971 A1 and 2003/0199963 A1, both filed by Tower, et al., describe a valved segment of bovine jugular vein, mounted within an expandable stent, for use as a replacement pulmonary valve. The replacement valve is mounted on a balloon catheter and delivered percutaneously via the vascular system to the location of the failed pulmonary valve and expanded by the balloon to compress the valve leaflets against the right ventricular outflow tract, anchoring and sealing the replacement valve. As described in the articles: “Percutaneous Insertion of the Pulmonary Valve”, Bonhoeffer, et al., Journal of the American College of Cardiology 2002; 39: 1664-1669 and “Transcatheter Replacement of a Bovine Valve in Pulmonary Position”, Bonhoeffer, et al., Circulation 2000; 102: 813-816, the replacement pulmonary valve may be implanted to replace native pulmonary valves or prosthetic pulmonary valves located in valved conduits.

Various types and configurations of prosthetic heart valves are used in percutaneous valve procedures to replace diseased natural human heart valves. The actual shape and configuration of any particular prosthetic heart valve is dependent to some extent upon the valve being replaced (i.e., mitral valve, tricuspid valve, aortic valve, or pulmonary valve). In general, the prosthetic heart valve designs attempt to replicate the function of the valve being replaced and thus will include valve leaflet-like structures used with either bioprostheses or mechanical heart valve prostheses.

That is, the replacement valves may include a valved vein segment that is mounted in some manner within an expandable stent to make a stented valve. In order to prepare such a valve for percutaneous implantation, the stented valve can be initially provided in an expanded or uncrimped condition, then crimped or compressed around the balloon portion of a catheter until it is as close to the diameter of the catheter as possible.

Other percutaneously-delivered prosthetic heart valves have been suggested having a generally similar configuration, such as by Bonhoeffer, P. et al., “Transcatheter Implantation of a Bovine Valve in Pulmonary Position.” Circulation, 2002; 102:813-816, and by Cribier, A. et al. “Percutaneous Transcatheter Implantation of an Aortic Valve Prosthesis for Calcific Aortic Stenosis.” Circulation, 2002; 106:3006-3008, the disclosures of which are incorporated herein by reference. These techniques rely at least partially upon a frictional type of engagement between the expanded support structure and the native tissue to maintain a position of the delivered prosthesis, although the stents can also become at least partially embedded in the surrounding tissue in response to the radial force provided by the stent and any balloons used to expand the stent. Thus, with these transcatheter techniques, conventional sewing of the prosthetic heart valve to the patient's native tissue is not necessary. Similarly, in an article by Bonhoeffer, P. et al. titled “Percutaneous Insertion of the Pulmonary Valve.” J Am Coll Cardiol, 2002; 39:1664-1669, the disclosure of which is incorporated herein by reference, percutaneous delivery of a biological valve is described. The valve is sutured to an expandable stent within a previously implanted valved or non-valved conduit, or a previously implanted valve. Again, radial expansion of the secondary valve stent is used for placing and maintaining the replacement valve.

Although there have been advances in percutaneous valve replacement techniques and devices, there is a continued desire to provide delivery systems and corresponding valves having features that allow for valve implantation in a minimally invasive and percutaneous manner.

### SUMMARY

The delivery systems and replacement valves of the invention are configured to provide complimentary features that promote optimal placement of the replacement heart valve in a native heart valve, such as the aortic valve, mitral valve, pulmonic valve, and/or tricuspid valve. In some embodiments, the replacement heart valves of the invention are highly amenable to transvascular delivery using a transapical approach (either with or without cardiopulmonary bypass and either with or without rapid pacing). The methodology associated with the present invention can be repeated multiple times, such that several prosthetic heart valves of the present invention can be mounted on top of or within one another, if necessary or desired.

Heart valves that can be placed in a patient using delivery systems of the invention include a stent to which a valve structure is attached. The stents can include a wide variety of structures and features that can be used alone or in combination with features of other stents. In particular, these stents provide a number of different docking and/or anchoring structures that are conducive to percutaneous delivery thereof. Many of the structures are thus compressible to a relatively small diameter for percutaneous delivery to the heart of the patient, and then are expandable either via removal of external compressive forces (e.g., self-expanding stents), or through application of an outward radial force (e.g., balloon expandable stents). The devices delivered by the delivery systems described herein can be used to deliver stents, valved stents, or other interventional devices such as ASD (atrial septal defect) closure devices, VSD (ventricular septal defect) closure devices, or PFO (patent foramen ovale) occluders.

Methods for insertion of the replacement heart valves of the invention include delivery systems that can maintain the stent structures in their compressed state during their insertion and allow or cause the stent structures to expand once they are in their desired location. In addition, some delivery methods of the invention can include features that allow the stents to be retrieved for removal or relocation thereof after they have been deployed or partially deployed from the stent delivery systems. The methods may include implantation of the stent structures using either an antegrade or retrograde approach. Further, in many of the delivery approaches of the invention, the stent structure is rotatable in vivo to allow the stent structure to be positioned in a desired orientation.

### BRIEF DESCRIPTION OF THE DRAWINGS

The present invention will be further explained with reference to the appended Figures, wherein like structure is referred to by like numerals throughout the several views, and wherein:

FIG. 1 is a front view of a portion of a stent positioned on a portion of an exemplary delivery system including multiple extending hubs or extending elements;

FIG. 2 is an enlarged view of a portion of the stent and delivery system illustrated in FIG. 1 and further illustrating a portion of a sheath positioned over the hubs and crowns of the stent;

FIG. 3 is a front view of a stent positioned relative to a distal portion of a delivery system and schematically positioned relative to a patient's anatomy, where the stent is being delivered using a retrograde approach;

FIG. 4 is a perspective view of a portion of another exemplary delivery system of the invention;

FIG. 5 is a perspective view of a stent positioned on the portion of the delivery system illustrated in FIG. 4 and further including a collar located over the crowns of the stent;

FIG. 6 is a perspective view of the portion of a delivery system and stent illustrated in FIG. 5, but with the collar in a different position relative to the other components of the delivery system;

FIG. 7 is a perspective view of a portion of another exemplary delivery system of the invention;

FIG. 8 is a perspective view of the portion of a delivery system illustrated in FIG. 7 and further illustrating a portion of a stent positioned on multiple hubs or extending members of the delivery system;

FIG. 9 is a side view of a delivery system that includes the portion of the delivery system illustrated in FIGS. 7 and 8, and further including a stent engaged with extending elements of the delivery system;

FIG. 10 is a cross-sectional view taken along section line A-A of the delivery system and stent illustrated in FIG. 9;

FIG. 11 is an enlarged view of the circled portion of the delivery system of FIG. 10;

FIG. 12 is a perspective view of a portion of another delivery system of the invention, including a stent that is engaged with features of the delivery system;

FIG. 13 is a schematic cross-sectional view of a portion of the delivery system of FIG. 12;

FIG. 14 is a front view of a stent positioned relative to a distal portion of a delivery system and schematically positioned relative to a patient's anatomy, wherein the stent is being delivered using an antegrade approach; and

FIG. 15 is a front view of another stent being delivered to a patient's anatomy using an antegrade approach.

### DETAILED DESCRIPTION

As referred to herein, the prosthetic heart valves used in accordance with the various devices and methods may include a wide variety of different configurations, such as a prosthetic heart valve having tissue leaflets or a synthetic heart valve having polymeric, metallic, or tissue-engineered leaflets, and can be specifically configured for replacing any heart valve. That is, while much of the description herein refers to replacement of aortic valves, the prosthetic heart valves of the invention can also generally be used for replacement of native mitral, pulmonic, or tricuspid valves, for use as a venous valve, or to replace a failed bioprosthesis, such as in the area of an aortic valve or mitral valve, for example.

Although each of the valves used with the delivery devices and methods described herein would typically include leaflets attached within an interior area of a stent, the leaflets are not shown in many of the illustrated embodiments for clarity purposes. In general, the stents described herein include a support structure comprising a number of strut or wire portions arranged relative to each other to provide a desired compressibility and strength to the heart valve. Other details on particular configurations of the stents of the invention are also described below; however, in general terms, stents of the invention are generally tubular support structures, and leaflets will be secured to the support structure. The leaflets can be formed from a variety of materials, such as autologous tissue, xenograph material, or synthetics as are known in the art. The leaflets may be provided as a homogenous, biological valve structure, such as a porcine, bovine, or equine valve. Alternatively, the leaflets can be provided independent of one another (e.g., bovine or equine pericardial leaflets) and subsequently assembled to the support structure of the stent. In another alternative, the stent and leaflets can be fabricated at the same time, such as may be accomplished using high strength nano-manufactured NiTi films produced at Advanced Bio Prosthetic Surfaces (ABPS), for example. The support structures are generally configured to accommodate three leaflets; however, the replacement prosthetic heart valves of the invention can incorporate more or less than three leaflets.

In more general terms, the combination of a support structure with one or more leaflets can assume a variety of other configurations that differ from those shown and described, including any known prosthetic heart valve design. In certain embodiments of the invention, the support structure with leaflets can be any known expandable prosthetic heart valve configuration, whether balloon expandable, self-expanding, or unfurling (as described, for example, in U.S. Pat. Nos. 3,671,979; 4,056,854; 4,994,077; 5,332,402; 5,370,685; 5,397,351; 5,554,185; 5,855,601; and 6,168,614; U.S. Patent Application Publication No. 2004/0034411; Bonhoeffer P., et al., “Percutaneous Insertion of the Pulmonary Valve”, Pediatric Cardiology, 2002; 39:1664-1669; Anderson H R, et al., “Transluminal Implantation of Artificial Heart Valves”, EUR Heart J., 1992; 13:704-708; Anderson, J. R., et al., “Transluminal Catheter Implantation of New Expandable Artificial Cardiac Valve”, EUR Heart J., 1990, 11: (Suppl) 224a; Hilbert S. L., “Evaluation of Explanted Polyurethane Trileaflet Cardiac Valve Prosthesis”, J Thorac Cardiovascular Surgery, 1989; 94:419-29; Block P C, “Clinical and Hemodynamic Follow-Up After Percutaneous Aortic Valvuloplasty in the Elderly”, The American Journal of Cardiology, Vol. 62, Oct. 1, 1998; Boudjemline, Y., “Steps Toward Percutaneous Aortic Valve Replacement”, Circulation, 2002; 105:775-558; Bonhoeffer, P., “Transcatheter Implantation of a Bovine Valve in Pulmonary Position, a Lamb Study”, Circulation, 2000:102:813-816; Boudjemline, Y., “Percutaneous Implantation of a Valve in the Descending Aorta In Lambs”, EUR Heart J, 2002; 23:1045-1049; Kulkinski, D., “Future Horizons in Surgical Aortic Valve Replacement: Lessons Learned During the Early Stages of Developing a Transluminal Implantation Technique”, ASAIO J, 2004; 50:364-68; the teachings of which are all incorporated herein by reference).

Orientation and positioning of the stents of the invention may be accomplished either by self-orientation of the stents (such as by interference between features of the stent and a previously implanted stent or valve structure) or by manual orientation of the stent to align its features with anatomical or previous bioprosthetic features, such as can be accomplished using fluoroscopic visualization techniques, for example. For example, when aligning the stents of the invention with native anatomical structures, they should be aligned so as to not block the coronary arteries, and native mitral or tricuspid valves should be aligned relative to the anterior leaflet and/or the trigones/commissures.

Some embodiments of the support structures of the stents described herein can be a series of wires or wire segments arranged so that they are capable of transitioning from a collapsed state to an expanded state. In some embodiments, a number of individual wires comprising the support structure can be formed of a metal or other material. These wires are arranged in such a way that a support structure allows for folding or compressing to a contracted state in which its internal diameter is greatly reduced from its internal diameter in an expanded state. In its collapsed state, such a support structure with attached valves can be mounted over a delivery device, such as a balloon catheter, for example. The support structure is configured so that it can be changed to its expanded state when desired, such as by the expansion of a balloon catheter. The delivery systems used for such a stent should be provided with degrees of rotational and axial orientation capabilities in order to properly position the new stent at its desired location.

The wires of the support structure of the stents in other embodiments can alternatively be formed from a shape memory material such as a nickel titanium alloy (e.g., Nitinol). With this material, the support structure is self-expandable from a contracted state to an expanded state, such as by the application of heat, energy, and the like, or by the removal of external forces (e.g., compressive forces). This support structure can also be compressed and re-expanded multiple times without damaging the structure of the stent. In addition, the support structure of such an embodiment may be laser cut from a single piece of material or may be assembled from a number of different components. For these types of stent structures, one example of a delivery system that can be used includes a catheter with a retractable sheath that covers the stent until it is to be deployed, at which point the sheath can be retracted to allow the stent to expand. Further details of such embodiments are discussed below.

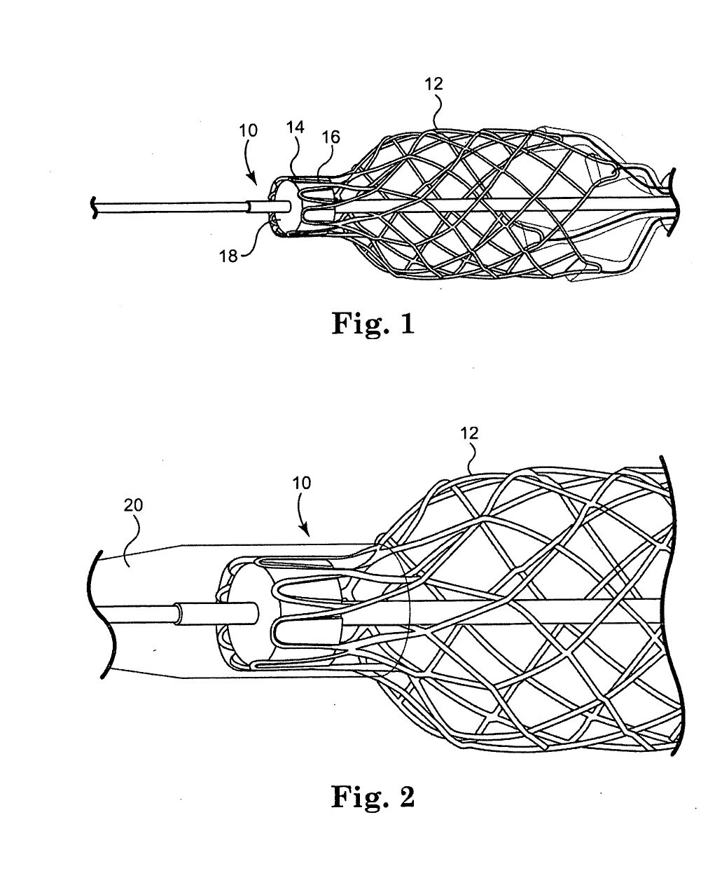

Referring now to the Figures, wherein the components are labeled with like numerals throughout the several Figures, and initially to FIGS. 1 and 2, a portion of a delivery system 10 is illustrated as engaged with a stent 12. In particular, the delivery system 10 includes multiple hubs 16 that extend from a central base portion 18. These extending hubs 16 are shaped for engagement with the crowns 14 of a distal end of the stent 12 to aid in securing the stent 12 to the delivery system 10. As shown, the crowns 14 of the stent 12 can “hook” over the hubs 16, which are generally shaped to match or engage with the compressed inner shape of the stent crowns 14. Thus, the number of hubs 16 that extend from the base portion 18 is preferably the same as the number of crowns 14 on the stent 12 that is engaged with it; however, the number of hubs 16 and number of crowns 14 may be different.

The proximal end of the stent 12 can be secured to the delivery system 10 in a number of different ways, such as with wires having hook tips that engage with the crowns of proximal end of the stent 12, for example. In this particular example, angled wire tips or protrusions extend at an angle relative to their respective wires, where the angle between each tip and its respective wire can be approximately 90 degrees, for example, although it can be any angle that provides for engagement between the wires and the stent crowns.

In order to keep the crowns 14 of the stent 12 engaged with the hubs 16 at the distal end of the delivery device 10 until it is desired to release the stent, the delivery system 10 is provided with a distal tip 20 that is hollow at its proximal end. In this way, the proximal end of the tip 20 can slide over the base portion 18 and its extending hubs 16 over which stent crowns 14 are positioned, thereby maintaining the system components in this arrangement. In order to release the stent 12 from the delivery system 10, the tip 20 and base portion 18 can be moved relative to each other until the tip 20 no longer covers the stent crowns 14. The crowns 14 will then be able to move outwardly from the hubs 16 to disengage the stent 12 from the hubs 16.

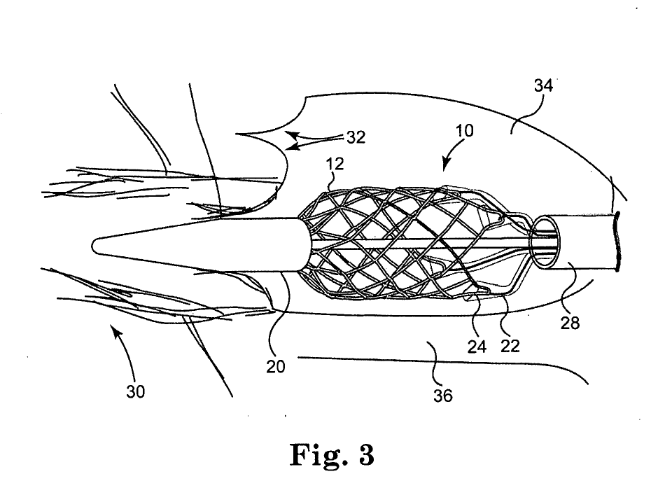

FIG. 3 illustrates one exemplary placement of the stent 12 for replacement of an aortic valve that uses the delivery system 10 having a distal end as shown in FIGS. 1 and 2. Alternatively, the delivery system 10 can be used for replacement of other valves and/or other portions of the body in which a stent is to be implanted. Regarding this schematic view of a portion of a patient's heart shown in FIG. 3, the delivery system 10 includes a tip 20 that maintains the stent 12 in a compressed condition at the distal end of the stent, wherein the crowns of the stent 12 (not visible in this Figure) at its distal end are hooked or positioned over the hubs of a sprocket. Alternatively, the crowns of the stent 12 at its distal end may be held in a compressed condition simply by the relatively smaller inner diameter of the tip 20 pressing the stent crowns toward the central axis of the delivery system 10 (i.e., no sprockets are used for this connection). The tip 20 is moveable relative to the crowns of the stent to compress, capture and release the stent crowns, as desired. The proximal end of the stent 12 is attached to the delivery system via multiple wires 22 that are engaged with the stent crowns at the proximal end of the stent 12. These wires 22 are further maintained on the stent crowns with sleeves 24 that surround or partially surround the wires 22 and their corresponding stent crowns. As shown, the area of the stent 12 between its proximal and distal ends is not compressed or held within any type of sheath and therefore is free to expand outwardly, as shown. However, it is contemplated that the stent 12 can be held in its compressed condition at various points during the stent delivery process using a sheath 28. The stent 12 is made of a series of wires that are compressible and expandable through the application and removal of external forces, and may include a series of Nitinol wires that are approximately 0.011-0.015 inches in diameter. That is, the stent 12 is a self-expanding stent.

The view of this area of a heart in FIG. 3 schematically illustrates an aorta 30, mitral valve 32, left ventricle 34, and septum 36. Further, the delivery approach illustrated in this Figure can be considered to be a retrograde delivery approach. In order to deploy the stent via the delivery system 10, the stent 12 is delivered to its desired position in a lumen (e.g. heart valve area) of a patient using a variety of tracking and monitoring techniques. In particular, FIG. 3 shows the stent and delivery system with the stent 12 being attached at both its proximal end and its distal end to the delivery system 10. When it is desired to release the stent 12, the stent is moved relative to the distal tip of the system, thereby exposing the distal end of the stent 12 and the stent crowns 14 that may be engaged with hubs 16. In this way, the compressive forces that were provided by enclosing the stent 12 within the tip 20 of the delivery system 10 are eliminated and the stent 12 can expand toward its original, expanded condition.

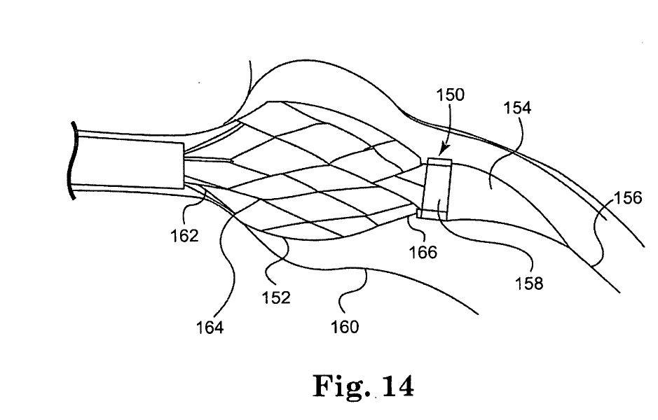

With the delivery system 10 of the invention, it is contemplated that the user can choose in which sequence the various portions of the stent are deployed or released while other portions are being held or compressed. For example, referring now to FIG. 14, a delivery system 150 is illustrated as it is being used to deliver a stent 152 to an aorta 160 using an antegrade approach. This may be performed either transapically or transeptally. Delivery system 150 includes a distal tip 154, a guidewire 156, a slideable sleeve 158, and attachment elements 162. With this configuration, the proximal or inflow end 164 of the stent 152 is attached to elements 162 which may be wires having hooks or coils and their distal ends, for example. Further, for antegrade delivery using this system, the proximal end 164 of the stent 152 can be released first while maintaining an outflow or distal end 166 of the stent 152 in a compressed state until it is determined that the stent is properly aligned and positioned relative to the native annulus of the aorta 160. The distal end 166 of the stent may then be released, such as by retracting the slideable sleeve 158 until the stent crowns at the distal end 166 are exposed and can thereby move relative to the delivery system 150.

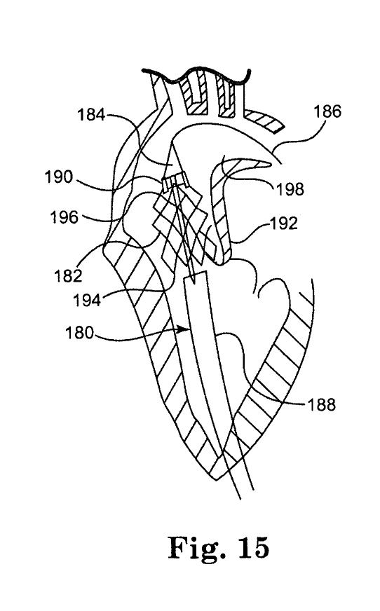

Another delivery system 180 is illustrated in FIG. 15 as it is being used to deliver a stent 182 to an aorta 198 using an antegrade approach. This may be performed either transapically or transeptally. Delivery system 180 includes a distal tip 184, a guidewire 186, a slideable sleeve 190, and an outer sheath 188. With this configuration, a portion of the delivery system 180 may include a sprocket with extending hubs (not visible) that can be positioned under the proximal end of the tip 184 to capture the crowns at the distal end 196 of the stent 182. When using this system for antegrade delivery of the stent 182 to the area of a native aortic valve 192, a proximal end 194 of the stent 182 can be fully released first, while maintaining the outflow or distal end 196 of the stent 182 in a compressed state. The system can remain in this arrangement until it is determined that the stent 182 is properly aligned and positioned relative to the native annulus of the aorta 192. The distal end 196 of the stent 182 can then be released, such as by retracting the slideable sleeve until the distal end of the stent 182 is exposed, thereby allowing the stent crowns to disengage from the sprocket with extending hubs.

It is noted that in the above procedure, the stent can be retracted back into a lumen of the delivery system 10 at any point in the process until the wires are disengaged from the stent, such as for repositioning of the stent if it is determined that the stent has been improperly positioned relative to the patient's anatomy. In this case, the steps described above can be repeated until the desired positioning of the stent is achieved.

With the delivery systems described herein, full or partial blood flow through the valve can advantageously be maintained during the period when the stented valve is being deployed into the patient but is not yet released from its delivery system. This feature can help to prevent complications that may occur when blood flow is stopped or blocked during valve implantation with some other known delivery systems. In addition, it is possible for the clinician to thereby evaluate the opening and closing of leaflets, examine for any paravalvular leakage and evaluate coronary flow and proper positioning of the valve within the target anatomy before final release of the stented valve.

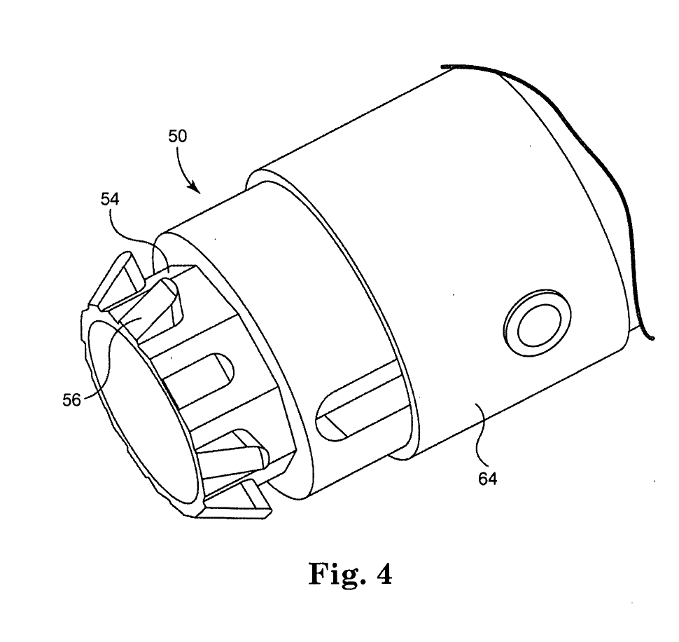

FIGS. 4-6 illustrate the distal end of another exemplary delivery system 50 that can be used to deploy a stent 52 in a desired location in a patient. The stent 52 includes a series of wires or wire segments arranged so that they are capable of transitioning from a collapsed state to an expanded state, and is preferably a self-expanding stent comprising a shape-memory material. The delivery system 50 is configured so that a stent can be loaded onto the system, at least partially deploying the stent, and then retracting the stent back into the delivery system and relocating it, if desired. In particular, delivery system 50 generally includes a base portion 54 that has multiple elements 56 extending from its outer, generally cylindrical surface. In one embodiment, the outer surface of the base portion 54 comprises multiple flat surfaces around its periphery, with one element 56 extending from each of the flat surfaces. In another embodiment, the outer surface of the base portion 54 has a generally circular outer periphery. The number of extending elements 56 preferably is the same as the number of wire structures 60 that extend from a proximal end of the stent 52, although the number of elements 56 can be different than the number of wire structures 60. Each element 56 comprises an angled wedge that tapers from its proximal end toward the distal end of the delivery system 50. All of the elements 56 of a particular delivery system 50 can be the same size and shape as each other, or the elements may be differently sized and shaped, as may be desirable for engagement with features of a particular stent.

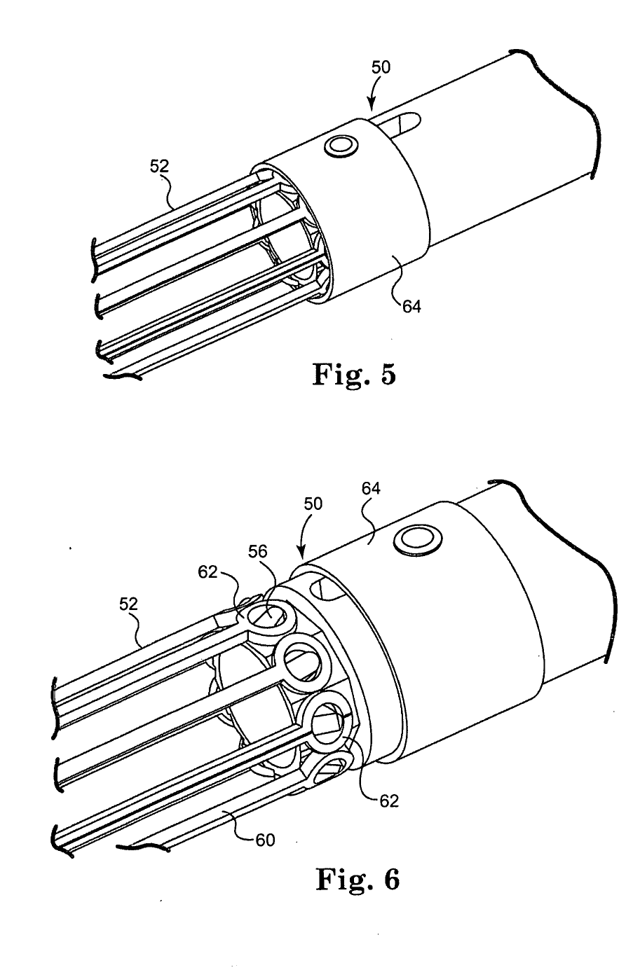

Stent 52 further includes a loop or eyelet 62 at the end of each wire structure 60, where each eyelet 62 is sized and shaped for engagement with an extending element 56. All of the eyelets 62 of a particular stent 60 may be the same size and shape as each other, or the eyelets 62 may be differently sized and shaped. In an alternative embodiment, the stent may include crowns at one end that are engageable with the extending elements 56, such as would be the case if the stent did not include eyelets at the end of its wire structures. Once the eyelets 62 (and/or stent crowns) are engaged with their respective elements 56 of the delivery system, a collar 64 can be slid at least partially over the eyelets 62, as is best illustrated in FIG. 5. When it is desired to release the stent eyelets 62 from the extending elements 56 of the delivery system 50, the collar 64 can be slid proximally to expose the eyelets 62 and extending elements 56, as is shown in FIG. 6, for example. One advantage provided by this collar 64 is that the outer sheath of the delivery system can be completely withdrawn, thereby allowing expansion of the stent to assess acceptable positioning while maintaining precise control and capture of the stent 52 on the extending elements 56.

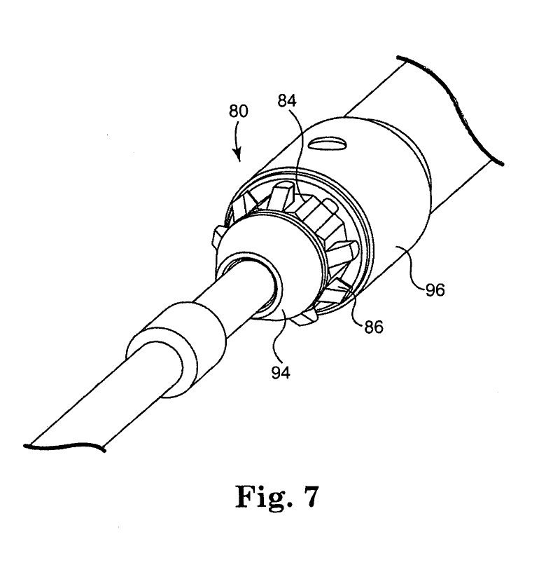

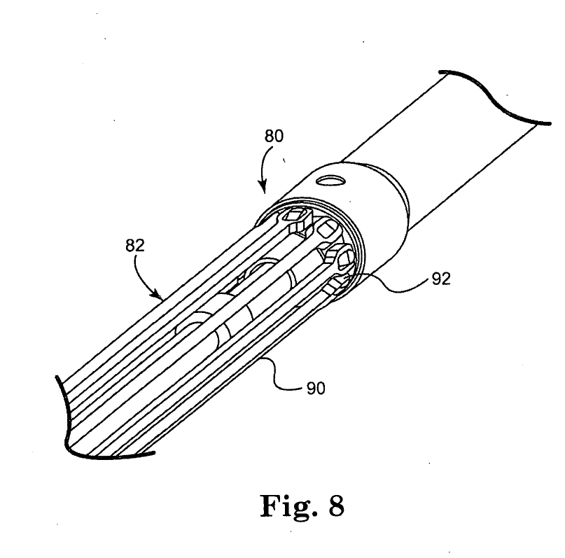

FIGS. 7 and 8 illustrate the distal end of another exemplary delivery system 80 that can be used to deploy a stent 82 in a desired location in a patient. The stent 82 includes a series of wires or wire segments arranged so that they are capable of transitioning from a collapsed state to an expanded state, and is preferably a self-expanding stent comprising a shape-memory material. The delivery system 80 is configured so that a stent can be loaded onto the system, at least partially deploying the stent, and then retracting the stent back into the delivery system and relocating it, if desired. In particular, delivery system 80 generally includes a base portion 84 that has multiple elements 86 extending from its outer, generally cylindrical surface. In one embodiment, the outer surface of the base portion 84 comprises multiple flat surfaces around its periphery, with one element 86 extending from each of the flat surfaces. In another embodiment, the outer surface of the base portion 84 has a generally circular outer periphery. The number of extending elements 86 preferably is the same as the number of wire structures 90 that extend from a proximal end of the stent 82, although the number of elements 86 can be different than the number of wire structures 90. Each element 86 comprises an angled wedge that tapers from its proximal end toward the distal end of the delivery system. All of the elements 86 of a particular delivery system 80 can be the same size and shape as each other, or the elements may be differently sized and shaped, as may be desirable for engagement with features of a particular stent. The delivery system 80 further includes a tapered nose portion 94 that extends from the sprocket 84 toward the distal end of the delivery system.

Stent 82 further includes a loop or eyelet 92 at the end of each wire structure 90, where each eyelet 92 is sized and shaped for engagement with an extending element 86. All of the eyelets 92 of a particular stent 90 may be the same size and shape as each other, or the eyelets 92 may be differently sized and shaped. In an alternative embodiment, the stent may include crowns at one end that are engageable with the extending elements 86, such as would be the case if the stent did not include eyelets at the end of its wire structures. The stent delivery system 80 further includes a spring-loaded collar 96 that can be configured so that it needs to be actuated to cover the eyelets 92 and the extending elements 86 with which they are engaged, or the collar 96 can be configured so that it needs to be actuated to uncover or release the eyelets 92. In either case, once the eyelets 92 (and/or stent crowns) are all engaged with their respective elements 86 of the delivery system, the collar 96 can be slid at least partially over the eyelets 92. When it is desired to release the stent eyelets 92 from the delivery system 80, the collar 96 can be slid proximally to expose the eyelets 92 and extending elements 86.

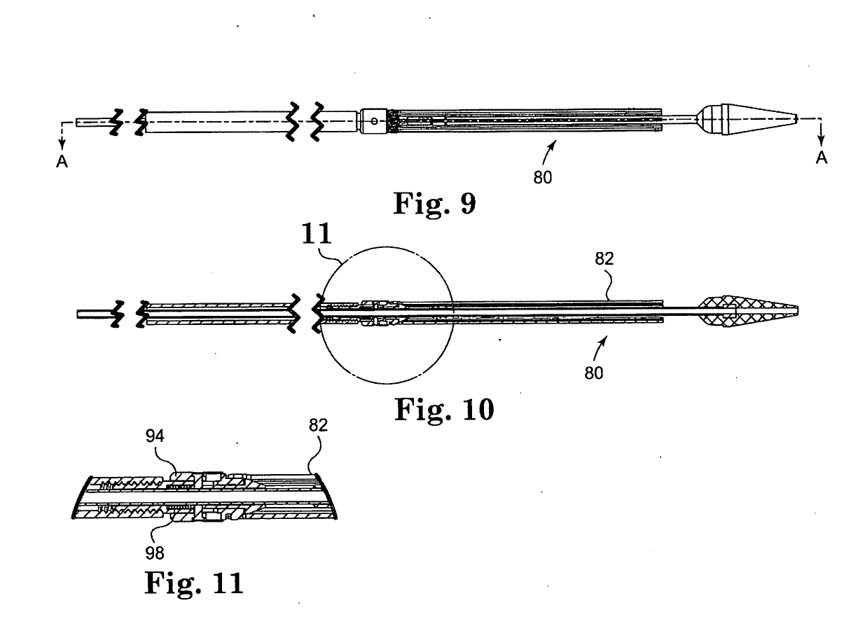

FIGS. 9-11 further illustrate one embodiment of the internal components and configuration of the delivery system 80 and a stent 82 engaged with the extending elements 86. This embodiment is shown without an outer sheath that can be used to maintain the stent in a compressed condition. One exemplary component that can be used for the spring-loaded collar 96 is a spring 98, although a different configuration for a spring can instead be used.

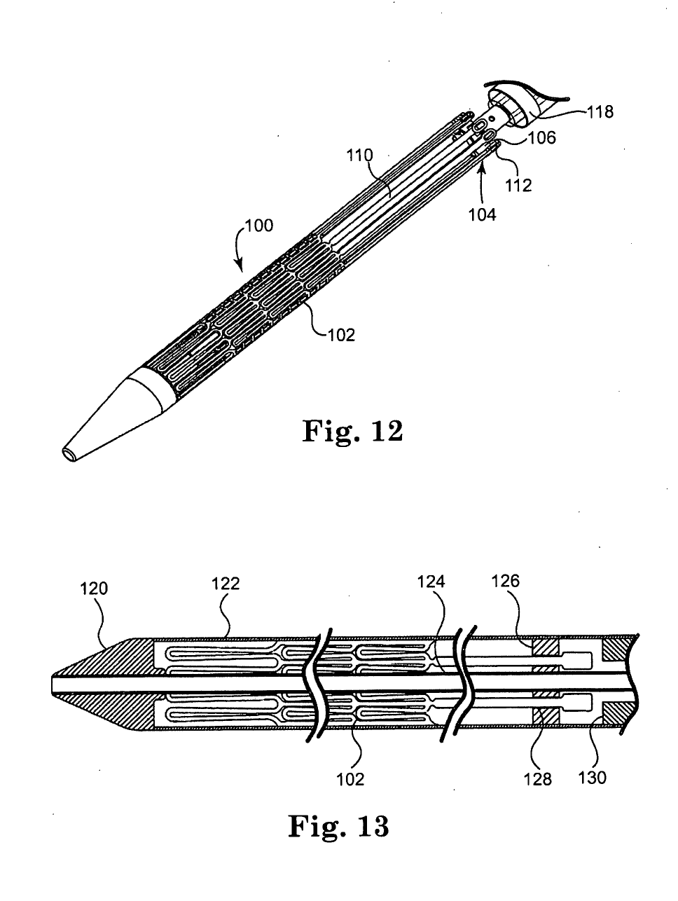

FIGS. 12 and 13 illustrate the distal end of another exemplary delivery system 100 that can be used to deliver and deploy a stent 102 in a desired location in a patient. The stent 102 includes a series of wires or wire segments arranged so that they are capable of transitioning from a collapsed state to an expanded state, and is preferably a self-expanding stent comprising a shape-memory material. The delivery system 100 is configured so that a stent can be loaded onto the system, at least partially deploying the stent, and then retracting the stent back into the delivery system and relocating it, if desired. In particular, delivery system 100 generally includes a sprocket 104 that includes multiple sprocket teeth 106 extending from its outer, generally cylindrical surface. The number of sprocket teeth 106 is preferably is the same as the number of wire structures 110 that extend from a proximal end of the stent 102, although the number of teeth 106 can be different than the number of wire structures 110. All of the teeth 106 of a particular delivery system 100 can be the same size and shape as each other, or the teeth 106 may be differently sized and shaped, as may be desirable for engagement with features of a particular stent.

Stent 102 further includes a loop or eyelet 112 at the end of each of its wire structures 110, where each eyelet 112 is sized and shaped for engagement with a sprocket tooth 106. All of the eyelets 112 of a particular stent may be the same size and shape as each other, or the eyelets 112 may be differently sized and shaped. In an alternative embodiment, the stent may include crowns at one end that are engageable with the teeth 106, such as would be the case if the stent did not include eyelets at the end of its wire structures.

The delivery system 100 further includes a sleeve 118 that is incorporated into the valve cover or sheath. In one exemplary embodiment, the delivery system will include a lock pin on its handle that would limit the travel of the valve cover to provide the additional control over the release of the stent, when desired.

FIG. 13 further illustrates a portion of the delivery system 100 in cross-section. In particular, delivery system 100 includes a catheter tip 120, an outer shaft 122, an inner member 124, an inner slotted ring 126, a slot 128, and a middle layer 130. A stent 102 is also illustrated in one exemplary manner in which it would be positioned on the delivery system 100 when in its compressed condition.

Although the number of extending elements, teeth, and/or hubs of the delivery systems described herein can vary, one preferred embodiment includes nine of such extending elements for engagement with a stent having nine attachment points. The stent attachment points can include stent crowns, eyelets at the end of stent crowns or at the end of extending stent wires, or the like. More or less than nine attachment points and extending elements can be provided; however, the size, shape, and spacing of the elements around a base portion can be adjusted accordingly to achieve a desired configuration and size for the overall delivery system. Further, the height of the various extending elements can be relatively large or small, depending on the thickness of the stent wires that will engage with it, the stresses to which the stent will be subjected during the delivery process, and the like. Because it is often desirable to minimize the diameter of the delivery system for percutaneous delivery of stented valves, the number of stent wires and corresponding extending elements can be designed or chosen to optimize the quality of the attachment between the stent and the delivery system, while providing a stent that has certain desirable characteristics when implanted in a patient.

With the various delivery systems of the invention, once the crowns, eyelets, or other stent features are engaged with sprocket teeth or other extending members of a delivery system, the stent can then be moved relative to a sheath of the delivery system to enclose the stent within the sheath by pulling the wires toward the proximal end of the delivery system, by pushing the sheath toward the distal end of the delivery system, or by some combination of these two movements. In order to release the stent once the delivery system is positioned within the patient, moving the sheath and stent in opposite directions relative to each other will provide the stent with the freedom to move. When the stent is a self-expanding stent, it can expand to a larger diameter once the constraint of the sheath is removed. Other motions can be performed to move the stent away from the teeth or engaging members with which it is engaged, if desired. The stent is thereby released from the delivery system.

One exemplary process for apically deploying a stent to the aorta using the delivery systems of the invention is described below. This method can also be used for other delivery approaches, such as for transarterial retrograde delivery. In particular, the stent is loaded onto the delivery system and a sheath is positioned over the stent to maintain it in its compressed condition. The delivery system is then advanced to the area in which the stent will be implanted using known delivery techniques and devices. Once the delivery system has been located within the patient so that the stent and/or valve is in its desired position within the aortic valve, the sheath is pulled back toward the proximal end of the delivery system (or the delivery system is moved distally away from the sheath), which allows the stent to expand radially. Alternatively, in a transarterial retrograde delivery approach, the inflow or annular end of the stent can remain compressed while the middle of the stent, along with other features such as petals, are radially expanded. Any minor positional adjustments, if necessary, can be made at this point. However, if it is determined that the stent is not in the desired position, the sheath can be moved back toward the distal end of the delivery system until the entire stent is again enclosed within the sheath, then the delivery system can be repositioned until it is in its desired location. The delivery system may also include a stop on its handle or some other portion of its structure that requires a positive action by the user to prevent inadvertent release of the stent from the delivery system.

Delivering any balloon-expandable stents to an implantation location can be performed percutaneously using modified versions of the delivery systems of the inventions. In general terms, this includes providing a transcatheter assembly, including a delivery catheter, a balloon catheter, and a guide wire. Some delivery catheters of this type are known in the art, and define a lumen within which the balloon catheter is received. The balloon catheter, in turn, defines a lumen within which the guide wire is slideably disposed. Further, the balloon catheter includes a balloon that is fluidly connected to an inflation source. It is noted that if the stent being implanted is the self-expanding type of stent, the balloon would not be needed and a sheath or other restraining means would be used for maintaining the stent in its compressed state until deployment of the stent, as described herein. In any case, for a balloon-expandable stent, the transcatheter assembly is appropriately sized for a desired percutaneous approach to the implantation location. For example, the transcatheter assembly can be sized for delivery to the heart valve via an opening at a carotid artery, a jugular vein, a sub-clavian vein, femoral artery or vein, or the like. Essentially, any percutaneous intercostals penetration can be made to facilitate use of the transcatheter assembly.

Prior to delivery, the stent is mounted over the balloon in a contracted state to be as small as possible without causing permanent deformation of the stent structure. As compared to the expanded state, the support structure is compressed onto itself and the balloon, thus defining a decreased inner diameter as compared to an inner diameter in the expanded state. While this description is related to the delivery of a balloon-expandable stent, the same basic procedures can also be applicable to a self-expanding stent, where the delivery system would not include a balloon, but would preferably include a sheath or some other type of configuration for maintaining the stent in a compressed condition until its deployment.

With the stent mounted to the balloon, the transcatheter assembly is delivered through a percutaneous opening (not shown) in the patient via the delivery catheter. The implantation location is located by inserting the guide wire into the patient, which guide wire extends from a distal end of the delivery catheter, with the balloon catheter otherwise retracted within the delivery catheter. The balloon catheter is then advanced distally from the delivery catheter along the guide wire, with the balloon and stent positioned relative to the implantation location. In an alternative embodiment, the stent is delivered to an implantation location via a minimally invasive surgical incision (i.e., non-percutaneously). In another alternative embodiment, the stent is delivered via open heart/chest surgery. In one embodiment of the stents of the invention, the stent includes a radiopaque, echogenic, or MRI visible material to facilitate visual confirmation of proper placement of the stent. Alternatively, other known surgical visual aids can be incorporated into the stent. The techniques described relative to placement of the stent within the heart can be used both to monitor and correct the placement of the stent in a longitudinal direction relative to the length of the anatomical structure in which it is positioned.

Once the stent is properly positioned, the balloon catheter is operated to inflate the balloon, thus transitioning the stent to an expanded state. Alternatively, where the support structure is formed of a shape memory material, the stent can self-expand to its expanded state.

The present invention has now been described with reference to several embodiments thereof. The foregoing detailed description and examples have been given for clarity of understanding only. No unnecessary limitations are to be understood therefrom. It will be apparent to those skilled in the art that many changes can be made in the embodiments described without departing from the scope of the invention. Thus, the scope of the present invention should not be limited to the structures described herein.

What is claimed is:

1. A delivery system for delivering a stented prosthetic heart valve to a lumen of a patient, the delivery system comprising:
a tubular body comprising a proximal end, a distal end, and a base portion comprising a plurality of extending elements, wherein each of the extending elements is engageable with a portion of a stent of a prosthetic heart valve; and
a tip portion comprising an inner area, the tip portion being longitudinally moveable relative to the base portion from a first position where the inner area at least partially covers the extending elements of the base portion to a second position where the extending elements are not positioned within the inner area of the tip portion.

2. The delivery system of claim 1, wherein the base portion is generally cylindrical.

3. The delivery system of claim 1, wherein each of the extending elements tapers in height from a first end toward a distal end of the delivery device.

4. The delivery system of claim 1, in combination with a stent having the same number of stent engagement features at a first end of the stent as the number of extending elements of the base portion.

5. The delivery system of claim 4, wherein the stent engagement features comprise a plurality of stent crowns at a first end of the stent.

6. The delivery system of claim 4, wherein the stent engagement features comprise a plurality of eyelets at the first end of the stent.

7. The delivery system of claim 4, wherein a second end of the stent includes a plurality of stent engagement features for engaging with a plurality of stent engagement devices of the delivery system.

8. The delivery system of claim 7, wherein the plurality of engagement features of the second end of the stent comprises a plurality of stent crowns at the second end of the stent.

9. The delivery system of claim 7, wherein the plurality of stent engagement devices of the delivery system comprise a plurality of wires that are engageable at their distal ends with the plurality of engagement features at the second end of the stent.

10. The delivery system of claim 7, wherein the stent engagement features of the second end of the stent are sequentially disengageable from the plurality of stent engagement devices prior to the stent engagement features of the first end of the stent being disengaged from the extending elements of the base portion.

11. The delivery system of claim 7, wherein the stent engagement features of the first end of the stent are sequentially disengageable from the extending elements of the base portion prior to the stent engagement features of the second end of the stent being disengaged from the plurality of stent engagement devices.

12. A delivery system for delivering a stented prosthetic heart valve to a lumen of a patient, the delivery system comprising:
a tubular body having a proximal end, a distal end, and a base portion comprising a plurality of extending elements, wherein each of the extending elements is engageable with a portion of a stent of a prosthetic heart valve; and
a sleeve comprising an inner area, the sheath being longitudinally moveable relative to the base portion from a first position where the inner area of the sleeve at least partially covers the extending elements of the base portion to a second position where the extending elements are not positioned within the inner area of the sleeve.

13. The delivery system of claim 12, wherein the sleeve is positioned at a proximal end of the stent.

14. The delivery system of claim 12, wherein the sleeve is spring-loaded for movement from a first position to a second position.
